(19) United States
(12) Patent Application Publication
(10) Pub. No.: US 2017/0143978 A1
(43) Pub. Date: May 25, 2017

(54) CONNECTOR FOR ELECTRICAL STIMULATION AND METHODS OF MAKING AND USING

(71) Applicant: Boston Scientific Neuromodulation Corporation, Valencia, CA (US)

(72) Inventor: John M. Barker, Thousand Oaks, CA (US)

(21) Appl. No.: 15/357,458

(22) Filed: Nov. 21, 2016

Related U.S. Application Data
(60) Provisional application No. 62/259,463, filed on Nov. 24, 2015.

(51) Int. Cl. A61N 1/375; A61N 1/36; A61N 1/05

(52) U.S. Cl. CPC A61N 1/3752; A61N 1/05; A61N 1/36071

(57) ABSTRACT

One embodiment is an electrical stimulation arrangement that includes a lead having a lead body and first contacts disposed along a first end surface of the proximal end portion of the lead body; and a lead extension having a lead extension body and second contacts disposed along the second end surface of the distal end portion of the lead extension body. Several of the first and second contacts form contact pairs with each contact pair containing one first contact and one second contact. For each contact pair, either the first contact or second contact is a male pin and the other of the first contact or second contact is a female socket configured and arranged to mate with the male pin when the proximal end portion of the lead body is mated to the distal end portion of the lead extension body forming a connector.

### CROSS-REFERENCE TO RELATED APPLICATIONS

This application claims the benefit under 35 U.S.C. §119(e) of U.S. Provisional Patent Application Ser. No. 62/259,463, filed Nov. 24, 2015, which is incorporated herein by reference.

### FIELD

The present invention is directed to the area of implantable electrical stimulation systems and methods of making and using the systems. The present invention is also directed to a connector for implantable electrical stimulation leads and lead extensions, as well as methods of making and using the connectors, leads, lead extensions, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an electrical stimulation arrangement that includes a lead having 1) a lead body having a distal end portion, a proximal end portion, and a first end surface on the proximal end portion of the lead body, 2) electrodes disposed along the distal end portion of the lead body, 3) first contacts disposed along the first end surface of the proximal end portion of the lead body, and 4) conductors coupling the first contacts to the electrodes; and a lead extension having 1) a lead extension body having a distal end portion, a proximal end portion, and a second end surface on the distal end portion of the lead extension body, 2) terminals disposed along the proximal end portion of the lead extension body, 3) second contacts disposed along the second end surface of the distal end portion of the lead extension body, and 4) conductors coupling the second contacts to the terminals. Several of the first and second contacts form contact pairs with each contact pair containing one of the first contacts and one of the second contacts. For each contact pair, either the first contact or second contact of the contact pair is a male pin and another of the first contact or second contact of the contact pair is a female socket configured and arranged to mate with the male pin when the proximal end portion of the lead body is mated to the distal end portion of the lead extension body forming a connector.

In at least some embodiments, the first contact of each contact pair is the male pin and the second contact of each contact pair is the female receptacle. In at least some embodiments, the first contacts are arranged in a first linear array and the second contacts are arranged in a second linear array. In at least some embodiments, the first contacts are arranged in a first two-dimensional array and the second contacts are arranged in a second two-dimensional array. In at least some embodiments, the electrical stimulation arrangement further includes a gasket disposed on each male pin or within each female socket. In at least some embodiments, the male pins and female sockets are configured and arranged to generate a tactile or audible response when mated. In at least some embodiments, the proximal end portion of the lead body and the distal end portion of the lead extension body, when mated to form the connector, have a same diameter as the lead body.

In at least some embodiments, the electrical stimulation arrangement further includes a sheath configured and arranged to slide over the connector formed by mating the proximal end portion of the lead body with the distal end portion of the lead extension body. In at least some embodiments, the sheath is an elastomeric sheath. In at least some embodiments, at least one of the lead body or the lead extension body includes a flange or groove to facilitate retention of the sleeve. In at least some embodiments, the sleeve includes a flange or groove to facilitate retention of the sleeve on the lead body or the lead extension body.

Another embodiment is an electrical stimulation system including the electrical stimulation arrangement described above; a control module coupleable to the lead extension, the control module including a housing, and an electronic subassembly disposed in the housing; and a connector for receiving the proximal end portion of the lead extension. The connector has a proximal end, a distal end, and a longitudinal length and the connector includes a connector housing defining a port at the distal end of the connector, the port configured and arranged for receiving the proximal end of the lead body of the electrical stimulation lead, and connector contacts disposed in the connector housing and configured and arranged to couple to the terminals disposed on the proximal end portion of the lead extension body of the lead extension.

Yet another embodiment is a method of implanting an electrical stimulation system. The method includes providing the electrical stimulation arrangement described above; implanting the lead; attaching the proximal end portion of the lead body to the distal end portion of the lead extension body to form the connector; and implanting the lead extension. In at least some embodiments, the method further includes coupling the lead extension to a control module. In at least some embodiments, the electrical stimulation arrangement further includes a sheath and the method further includes sliding the sheath over the connector. In at least some embodiments, the sheath is disposed on the lead extension prior to sliding the sheath over the connector.

A further embodiment is an electrical stimulation arrangement including a first elongate device having 1) a first body having a distal end portion, a proximal end portion, and a first end surface on the proximal end portion of the first body, 2) first contacts disposed along the first end surface of the proximal end portion of the first body, and 3) conductors electrically coupled to the first contacts and extending along the first body; and a second elongate device having 1) a second body having a distal end portion, a proximal end portion, and a second end surface on the distal end portion of the second body, 2) second contacts disposed along the second end surface of the distal end portion of the second body, and 3) conductors electrically coupled to the second contacts and extending along the second body. The first and second contacts form contact pairs with each contact pair containing one of the first contacts and one of the second contacts. For each contact pair, either the first contact or second contact of the contact pair is a male pin and another of the first contact or second contact of the contact pair is a female socket configured and arranged to mate with the male pin when the proximal end portion of the first body is mated to the distal end portion of the second body forming a connector.

In at least some embodiments, the electrical stimulation arrangement further includes a sheath configured and arranged to slide over the connector formed by mating the proximal end portion of the first body with the distal end portion of the second body. In at least some embodiments, the electrical stimulation arrangement further includes a gasket disposed on each male pin or within each female socket.

Yet another embodiment is an electrical stimulation arrangement including a first elongate device having 1) a first body having a first end surface, 2) first contacts disposed along the first end surface of the first body, and 3) conductors electrically coupled to the plurality of first contacts; and a second device having 1) a second body having a second end surface, 2) second contacts disposed along the second end surface of the second body, and 3) conductors electrically coupled to the plurality of second contacts. The first and second contacts form contact pairs with each contact pair containing one of the first contacts and one of the second contacts. For each contact pair, either the first contact or second contact of the contact pair is a male pin and another of the first contact or second contact of the contact pair is a female socket configured and arranged to mate with the male pin when the first end surface of the first body is mated to the second end surface of the second body forming a connector.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an electrical stimulation system that includes a paddle lead electrically coupled to a control module, according to the invention;

FIG. 2 is a schematic side view of one embodiment of an electrical stimulation system that includes a percutaneous lead electrically coupled to a control module, according to the invention;

FIG. 3A is a schematic side view of one embodiment of the control module of FIG. 1 configured and arranged to electrically couple to an elongated device, according to the invention;

FIG. 3B is a schematic side view of one embodiment of a lead extension configured and arranged to electrically couple the leads of FIGS. 1 and 2 to the control module of FIG. 1, according to the invention;

FIG. 4 is a schematic perspective view of one embodiment of a connector formed from the distal portion of a lead extension and proximal portion of lead prior to connection, according to the invention;

FIG. 5 is schematic cross-sectional view of another embodiment of a connector formed from the distal portion of a lead extension and proximal portion of lead after connection, according to the invention; and

FIG. 6 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to a connector for implantable electrical stimulation leads and lead extensions, as well as methods of making and using the connectors, leads, lead extensions, and electrical stimulation systems.

Suitable implantable electrical stimulation systems include, but are not limited to, a least one lead with one or more electrodes disposed along a distal end of the lead and one or more terminals disposed along the one or more proximal ends of the lead.

Leads include, for example, percutaneous leads, paddle leads, and cuff leads. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties.

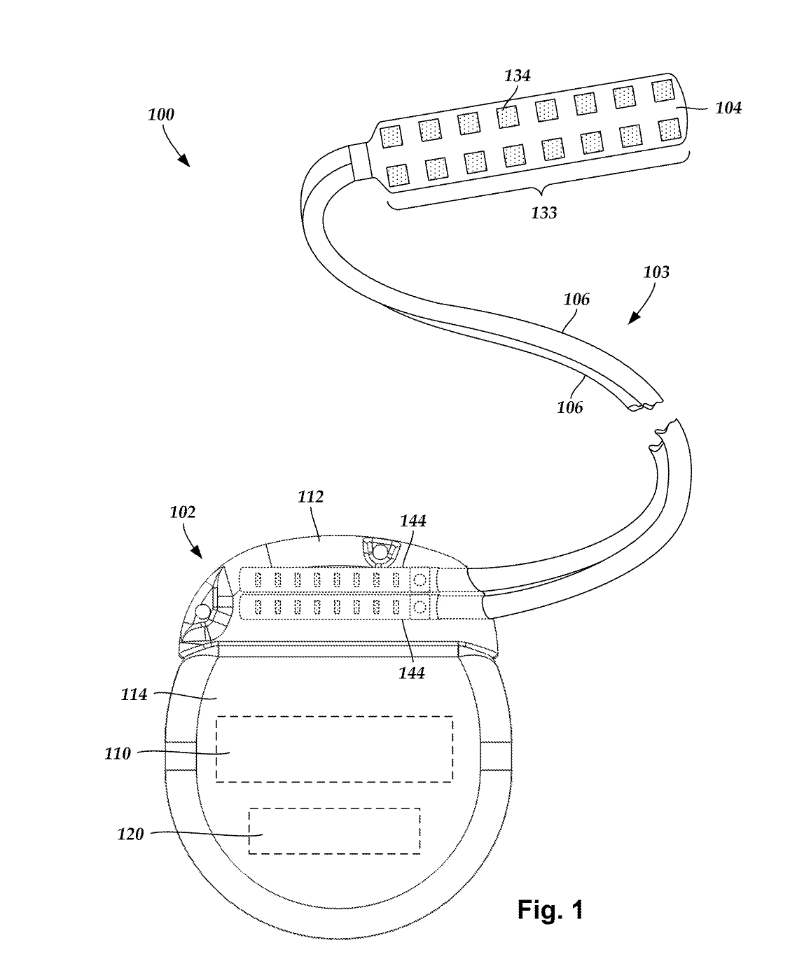

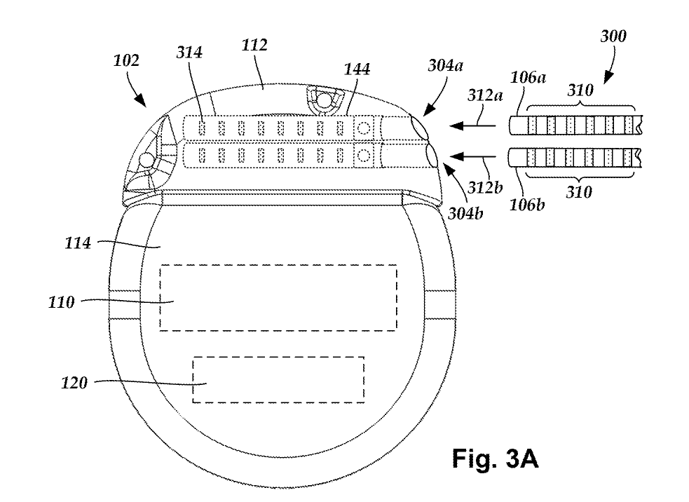

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array 133 of electrodes, such as electrode 134, is disposed on the paddle body 104, and an array of terminals (e.g., 310 in FIG. 3A) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

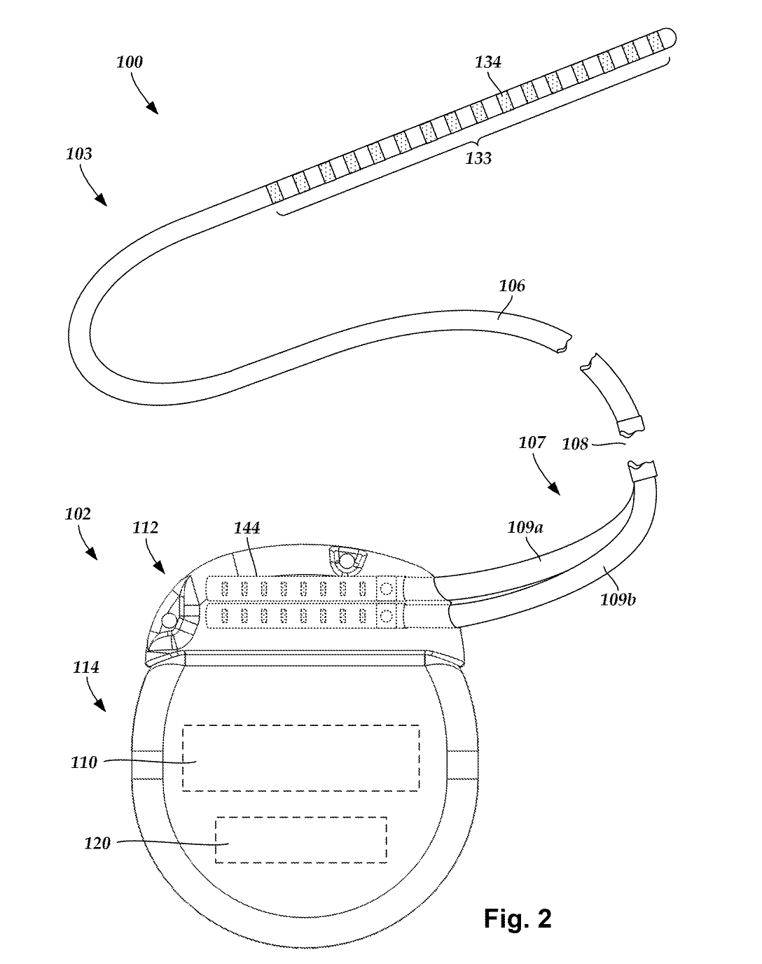

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices (324 in FIG. 3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more splitter tails 109a and 109b configured and arranged to couple to the control module 102 (or another splitter, a lead extension, an adaptor, or the like).

With reference to FIGS. 1 and 2, the control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

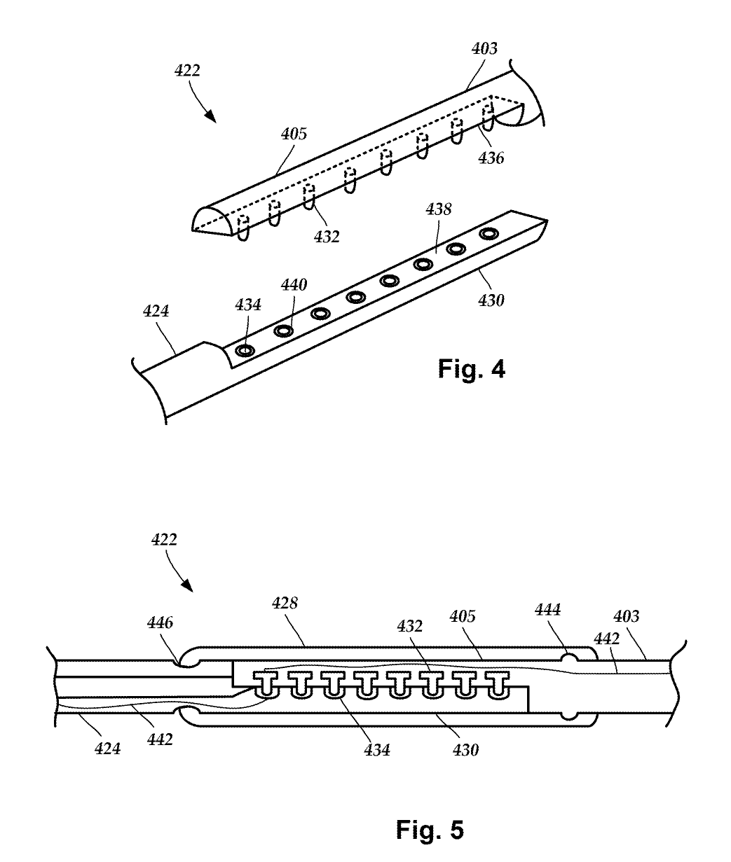

Terminals (e.g., 310 in FIG. 3A) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIG. 3A). The connector contacts are disposed in control modules connectors 144 which, in turn, are disposed on, for example, the control module 102 (or a splitter, an adaptor, or the like). Electrically conductive wires, cables, or the like (e.g., 442 in FIG. 5) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 3B is a schematic side view of an embodiment of an electrical stimulation system 100. The electrical stimulation system 100 includes a lead extension 324 that is configured and arranged to couple an elongated device 300 (e.g., one of the lead bodies 106 of FIGS. 1 and 2, the splitter 107 of FIG. 2, an adaptor, another lead extension, or the like or combinations thereof) to the control module 102. The lead extension 324 includes a lead extension body 325 similar to the lead body described above. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

The lead extension 324 and elongated device 300 couple together to form a connector 322 with an optional sleeve 328. The connector 322 is disposed at a distal end of the lead extension 324 and the proximal end of the elongated device 300.

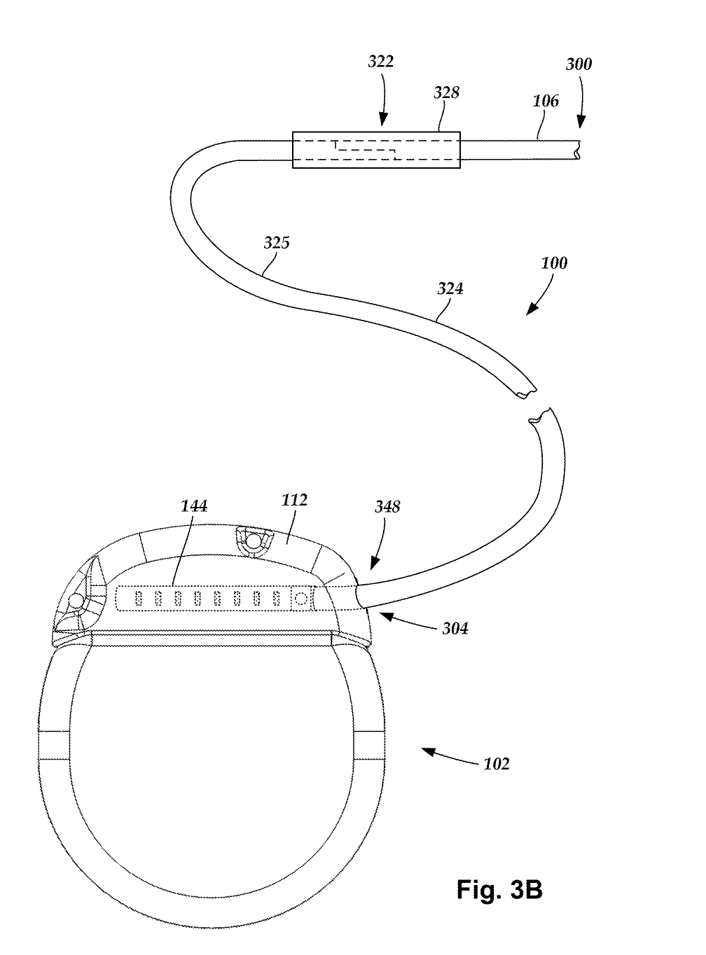

FIG. 4 illustrates one embodiment of a connector 422 formed from a distal portion of a lead extension 424 and a proximal portion of a lead 403. It will be understood that the lead extension and lead can be replaced by another other suitable components, such as, for example, another lead extension, an adapter, a splitter, any other elongated device, a control module or the like. The lead extension 424 and lead 403 are illustrated as examples for description of the connector 422.

A first connector portion 405 of the lead 403 and a second connector portion 430 of the lead extension 424 are shaped to physically overlap and mate with each other to form the connector 422. In at least some embodiments, the first connector portion 405 and the second connector portion 430 are shaped as cross cut half cylinders, although other shapes can be used. In at least some embodiments, the first connector portion 405 and the second connector portion 430, when physically mated, maintain the same outer diameter as both the lead 403 and lead extension 424, thereby providing a lower profile connector than conventional connector between a lead and a lead extension. In other embodiments, the mated connector 422 can have a diameter that is larger (or smaller) than one or both of the lead 403 and lead extension 424. In at least some embodiments, the first and second connector portions 405, 430 are flexible and may be more flexible than a connector of a conventional lead extension, thereby providing a more flexible connector between a lead and lead extension.

Electrical connection between the lead 403 and lead extension 424 is made through male pins 432 and female sockets 434 arrayed on the first connector portion 405 and the second connector portion 430. In the illustrated embodiment, the male pins 432 are arrayed on the first connector portion 405 and the female sockets 434 are arrayed on the second connector portion 430. In other embodiments, the male pins can be arrayed on the second connector portion and the female sockets can be arrayed on the first connector portion. In yet other embodiments, each of the first connector portion and second connector portion can include both male pins and female sockets. For example, the male pins and female sockets can alternate along each of the first connector portion and the second connector portion or either male pins or female sockets may be presented at the end of each of the connector portions with the opposite component (female sockets or male pins) disposed closer to the cylindrical portion of the lead or lead extension. Any other arrangement of male pins and female sockets can be used. In the illustrated embodiment, the male pins 432 are the terminals of the lead 403 (similar in electrical function to the terminals described above with respect to the lead 103 of FIGS. 1 and 2.)

In at least some embodiments, the male pins 432 and female sockets 434 are each arranged in a linear array, as illustrated in FIG. 4. In other embodiments, the male pins and female sockets can be arranged in a two-dimensional array. This may be particularly useful with larger numbers (for example, 10, 12, 14, 16, or more pins or sockets). In yet other embodiments, the male pins and female sockets can be arranged so that alternating pins/sockets are staggered. Any other regular, irregular, or random array of pins or sockets can also be used. The embodiment of FIG. 4 illustrates eight pins 432 and sockets 434, but it will be understood that any number of pins and sockets can be used including, but not limited to, 2, 4, 6, 10, 12, 14, 16, 18, 20, 32, or more. In at least some embodiments, the number of sockets 434 and pins 432 are the same. In other embodiments, there may be more sockets than pins.

In at least some embodiments, the male pins 432 and female sockets 434 are disposed on surfaces 436, 438 of the first connector portion 405 and the second connector portion 430, respectively, that will be within the interior of the connector 422 when the two portions 405, 430 are mated, as illustrated in FIG. 4. In at least some embodiments, these surfaces 436, 438 are flat, as illustrate in FIG. 4. Thus, in contrast to many conventional connectors, which have contacts arranged around a cylindrical surface, in at least some embodiments, the pins 432, and sockets 434 are disposed on flat surfaces. In addition, in at least some embodiments, the first connector portion 405 and the second connector portion 430, when joined to form the connector 422, the resulting connector has a cylindrical shape. For example, the first and second connector portions 405, 430 can be half cylinders, as illustrated in FIG. 4, that, when joined, form a cylindrical connector 422.

The connector 422 provides both an electrical and mechanical connection that supports retention of the lead 403 with the lead extension 424. In at least some embodiments, the connection between the lead 403 and lead extension 424 is physically retained by each of the male pin 432 to female socket 434 connector joints which are at a right angle to the axis of any tensile loading on this connection.

In at least some embodiments, electrical isolation and sealing from fluid of each of the male pin/female socket connections can be accomplished by incorporating and elastomer gasket 440 (for example, an O-ring) around each of the female sockets 434.

Alternatively, the gasket 440 can be disposed around the male pins 432, instead of (or in addition to) the female sockets 434. Alternatively or additionally, a gasket can surround the array of pins 432 or socket 434 or can be disposed around a perimeter of the surface 436 or surface 438 to provide a seal for the connector 422.

In at least some embodiments, the male pins 432 and female sockets 434 may include fastening elements that engage each other to facilitate retention of the pins within the sockets. For example, the male pins 432 and female sockets 434 can include one or more flanges on either the pins or sockets and corresponding detents on the other of the pins or sockets (or grooves and ridges on respective pins or sockets) to engage to facilitate retention of the pins within the sockets. In at least some embodiments, the engagement of the male pins 432 and female sockets 434 may provide a tactile or audible (or both) response to indicate to the practitioner that the pins and sockets are engaged. For example, the male pins 432 may snap into the female sockets 434 and provide a tactile or audible (or both) response.

Both the male pins 432 and female sockets 434 are electrically conductive (and can be collectively referred to as “contacts”) and, when mated, are electrically coupled to each other. The male pins 432 and female sockets 434 are also electrically coupled to conductors 442 (e.g., wires) that extend along the corresponding lead 403 or lead extension 424 to electrically couple to electrodes 134 (FIGS. 1 and 2) of the lead or terminals 310 (FIG. 3A) of the lead extension, respectively.

The connector 422 optionally includes a sleeve 428 that can be slid, or otherwise disposed, over the first connector portion 405 and the second connector portion 430 when mated, as illustrated in FIG. 5. The sleeve 428 is preferably non-conductive and is preferably elastomeric. In at least some embodiments, the sleeve 428 is molded of an elastomer material (such as silicone). The sleeve may provide a sealing interference fit with the joined lead and lead extension and may also support the retention of the joint.

In at least some embodiments, the sleeve 428 is mounted onto the distal end of the lead extension 424 (or, alternatively, on the proximal end of the lead 403) and can be advanced distally or retracted proximally over the first connector portion 405 and the second connector portion 430. In at least some embodiments, the sleeve 428 is mounted on the lead extension 424 to allow the lead 403 to remain isodiametric for passing completely through an insertion needle. In at least some embodiments, the sleeve 428 is sized to lock over, and capture, the lead and lead extension ends when they are mated together.

In at least some embodiments, the lead 402 or lead extension 424 (or both) include flanges 444 (for example, annular flanges) or grooves 446 (for example, annular grooves) or any combination thereof to facilitate retention of the sleeve 428 over the mated first connector portion 405 and second connector portion 430. Alternatively, the sleeve 428 may also include flanges or grooves or any combination thereof that can mate with flanges 444 or grooves 446 on the lead or lead extension. The illustrated embodiment include a flange 444 on the lead 403 and a groove on the lead extension 424, but it will be understood that any arrangement of one or more flanges or grooves (or combinations thereof) can be utilized.

Many conventional neurostimulation leads incorporate flush mounted cylindrical terminals (often 4 to 8) that are electrically coupled to distal electrodes at the distal end of a lead. The proximal end of the lead may be joined to a lead extension via a female port in a connector located at the end of the extension. This connector often incorporates spring contacts within the female port that allow each lead contact to be joined to corresponding cables and proximal contacts with the lead extension which, when the extension is connected to an implantable pulse generator, will complete a neurostimulation delivery system for treating pain or other medical conditions. This connector on the distal end of the lead extension also often includes features for locking the lead to lead extension, for example, a set screw and connector block at the distal end of the connector which allows locking to a mating metal retention sleeve located on the proximal end of the lead using a small torque wrench. In at least some instances, this conventional lead extension connector has a relatively large diameter compared to the lead and lead extension, can be bulky and mechanically complex due to the spring contacts and sets screws.

The connector 422, in at least some embodiments, can address one or more of these concerns related to conventional connectors. In at least some embodiments, the connector 422 can provide a lower profile than a conventional lead extension connector. In at least some embodiments, the connector 422 can provide a more flexible connection than a convention lead extension connector which can produce increased patient comfort and tissue acceptance of the implant. In at least some embodiments, the connector 422 can reduce manufacturing cost by eliminating the retention sleeve, connector block, set screw, septum, and spring contacts of the convention lead extension connector. In at least some embodiments, the connector 422 can eliminate the need for a torque wrench or other tool for locking the lead to lead extension (using, for example, a set screw.)

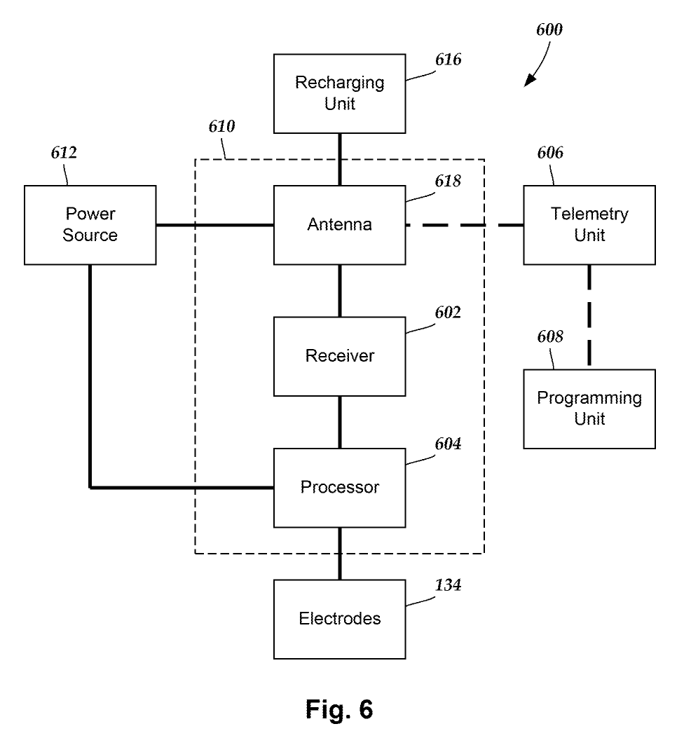

FIG. 6 is a schematic overview of one embodiment of components of an electrical stimulation system 600 including an electronic subassembly 610 disposed within a control module. It will be understood that the electrical stimulation system can include more, fewer, or different components and can have a variety of different configurations including those configurations disclosed in the stimulator references cited herein.

Some of the components (for example, a power source 612, an antenna 618, a receiver 602, and a processor 604) of the electrical stimulation system can be positioned on one or more circuit boards or similar carriers within a sealed housing of an implantable pulse generator, if desired. Any power source 612 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the optional antenna 618 or a secondary antenna. The external power source can be in a device that is mounted on the skin of the user or in a unit that is provided near the user on a permanent or periodic basis.

If the power source 612 is a rechargeable battery, the battery may be recharged using the optional antenna 618, if desired. Power can be provided to the battery for recharging by inductively coupling the battery through the antenna to a recharging unit 616 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 604 is generally included to control the timing and electrical characteristics of the electrical stimulation system. For example, the processor 604 can, if desired, control one or more of the timing, frequency, strength, duration, and waveform of the pulses. In addition, the processor 604 can select which electrodes can be used to provide stimulation, if desired. In some embodiments, the processor 604 selects which electrode(s) are cathodes and which electrode(s) are anodes. In some embodiments, the processor 604 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 608 that, for example, allows modification of pulse characteristics. In the illustrated embodiment, the processor 604 is coupled to a receiver 602 which, in turn, is coupled to the optional antenna 618. This allows the processor 604 to receive instructions from an external source to, for example, direct the pulse characteristics and the selection of electrodes, if desired.

In one embodiment, the antenna 618 is capable of receiving signals (e.g., RF signals) from an external telemetry unit 606 which is programmed by the programming unit 608. The programming unit 608 can be external to, or part of, the telemetry unit 606. The telemetry unit 606 can be a device that is worn on the skin of the user or can be carried by the user and can have a form similar to a pager, cellular phone, or remote control, if desired. As another alternative, the telemetry unit 606 may not be worn or carried by the user but may only be available at a home station or at a clinician's office. The programming unit 608 can be any unit that can provide information to the telemetry unit 606 for transmission to the electrical stimulation system 600. The programming unit 608 can be part of the telemetry unit 606 or can provide signals or information to the telemetry unit 606 via a wireless or wired connection. One example of a suitable programming unit is a computer operated by the user or clinician to send signals to the telemetry unit 606.

The signals sent to the processor 604 via the antenna 618 and the receiver 602 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 600 to cease operation, to start operation, to start charging the battery, or to stop charging the battery. In other embodiments, the stimulation system does not include the antenna 618 or receiver 602 and the processor 604 operates as programmed.

Optionally, the electrical stimulation system 600 may include a transmitter (not shown) coupled to the processor 604 and the antenna 618 for transmitting signals back to the telemetry unit 606 or another unit capable of receiving the signals. For example, the electrical stimulation system 600 may transmit signals indicating whether the electrical stimulation system 600 is operating properly or not or indicating when the battery needs to be charged or the level of charge remaining in the battery. The processor 604 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation arrangement, comprising:
a lead comprising 1) a lead body having a distal end portion, a proximal end portion, and a first end surface on the proximal end portion of the lead body, 2) a plurality of electrodes disposed along the distal end portion of the lead body, 3) a plurality of first contacts disposed along the first end surface of the proximal end portion of the lead body, and 4) a plurality of conductors coupling the plurality of first contacts to the plurality of electrodes; and
a lead extension comprising 1) a lead extension body having a distal end portion, a proximal end portion, and a second end surface on the distal end portion of the lead extension body, 2) a plurality of terminals disposed along the proximal end portion of the lead extension body, 3) a plurality of second contacts disposed along the second end surface of the distal end portion of the lead extension body, and 4) a plurality of conductors coupling the plurality of second contacts to the plurality of terminals;
wherein a plurality of the first and second contacts form contact pairs, each contact pair containing one of the first contacts and one of the second contacts, wherein, for each contact pair, either the first contact or second contact of the contact pair is a male pin and another of the first contact or second contact of the contact pair is a female socket configured and arranged to mate with the male pin when the proximal end portion of the lead body is mated to the distal end portion of the lead extension body forming a connector.

2. The electrical stimulation arrangement of claim 1, wherein the first contact of each contact pair is the male pin and the second contact of each contact pair is the female receptacle.

3. The electrical stimulation arrangement of claim 1, wherein the first contacts are arranged in a first linear array and the second contacts are arranged in a second linear array.

4. The electrical stimulation arrangement of claim 1, wherein the first contacts are arranged in a first two-dimensional array and the second contacts are arranged in a second two-dimensional array.

5. The electrical stimulation arrangement of claim 1, further comprising a gasket disposed on each male pin or within each female socket.

6. The electrical stimulation arrangement of claim 1, wherein the male pins and female sockets are configured and arranged to generate a tactile or audible response when mated.

7. The electrical stimulation arrangement of claim 1, further comprising a sheath configured and arranged to slide over the connector formed by mating the proximal end portion of the lead body with the distal end portion of the lead extension body.

8. The electrical stimulation arrangement of claim 7, wherein the sheath is an elastomeric sheath.

9. The electrical stimulation arrangement of claim 7, wherein at least one of the lead body or the lead extension body comprises a flange or groove to facilitate retention of the sleeve.

10. The electrical stimulation arrangement of claim 9, wherein the sleeve comprises a flange or groove to facilitate retention of the sleeve on the lead body or the lead extension body.

11. The electrical stimulation arrangement of claim 1, wherein the proximal end portion of the lead body and the distal end portion of the lead extension body, when mated to form the connector, have a same diameter as the lead body.

12. An electrical stimulation system comprising:
the electrical stimulation arrangement of claim 1;
a control module coupleable to the lead extension, the control module comprising
a housing, and
an electronic subassembly disposed in the housing; and
a connector for receiving the proximal end portion of the lead extension, the connector having a proximal end, a distal end, and a longitudinal length, the connector comprising
a connector housing defining a port at the distal end of the connector, the port configured and arranged for receiving the proximal end of the lead body of the electrical stimulation lead, and
a plurality of connector contacts disposed in the connector housing, the plurality of connector contacts configured and arranged to couple to the terminals disposed on the proximal end portion of the lead extension body of the lead extension.

13. A method of implanting an electrical stimulation system, the method comprising:
providing the electrical stimulation arrangement of claim 1;
implanting the lead;
attaching the proximal end portion of the lead body to the distal end portion of the lead extension body to form the connector; and
implanting the lead extension.

14. The method of claim 13, further comprising coupling the lead extension to a control module.

15. The method of claim 13, wherein the electrical stimulation arrangement further comprises a sheath and the method further comprising sliding the sheath over the connector.

16. The method of claim 15, wherein the sheath is disposed on the lead extension prior to sliding the sheath over the connector.

17. An electrical stimulation arrangement, comprising:
a first elongate device comprising 1) a first body having a distal end portion, a proximal end portion, and a first end surface on the proximal end portion of the first body, 2) a plurality of first contacts disposed along the first end surface of the proximal end portion of the first body, and 3) a plurality of conductors electrically coupled to the plurality of first contacts and extending along the first body; and
a second elongate device comprising 1) a second body having a distal end portion, a proximal end portion, and a second end surface on the distal end portion of the second body, 2) a plurality of second contacts disposed along the second end surface of the distal end portion of the second body, and 3) a plurality of conductors electrically coupled to the plurality of second contacts and extending along the second body;
wherein a plurality of the first and second contacts form contact pairs, each contact pair containing one of the first contacts and one of the second contacts, wherein, for each contact pair, either the first contact or second contact of the contact pair is a male pin and another of the first contact or second contact of the contact pair is a female socket configured and arranged to mate with the male pin when the proximal end portion of the first body is mated to the distal end portion of the second body forming a connector.

18. The electrical stimulation arrangement of claim 17, further comprising a sheath configured and arranged to slide over the connector formed by mating the proximal end portion of the first body with the distal end portion of the second body.

19. The electrical stimulation arrangement of claim 17, further comprising a gasket disposed on each male pin or within each female socket.

20. An electrical stimulation arrangement, comprising:
a first elongate device comprising 1) a first body having a first end surface, 2) a plurality of first contacts disposed along the first end surface of the first body, and 3) a plurality of conductors electrically coupled to the plurality of first contacts; and
a second device comprising 1) a second body having a second end surface, 2) a plurality of second contacts disposed along the second end surface of the second body, and 3) a plurality of conductors electrically coupled to the plurality of second contacts;
wherein a plurality of the first and second contacts form contact pairs, each contact pair containing one of the first contacts and one of the second contacts, wherein, for each contact pair, either the first contact or second contact of the contact pair is a male pin and another of the first contact or second contact of the contact pair is a female socket configured and arranged to mate with the male pin when the first end surface of the first body is mated to the second end surface of the second body forming a connector.
